(19) United States
(12) Patent Application Publication
(10) Pub. No.: US 2003/0215499 A1
(43) Pub. Date: Nov. 20, 2003

(54) Use of completely linear short chain alpha-glucans as a pharmaceutical excipient

(76) Inventors: Yong-Cheng Shi, Hillsborough, NJ (US); Xiaoyuan Cui, Belle Mead, NJ (US); Sibu Chakrabarti, Randolph, NJ (US)

Correspondence Address: Karen G. Kaiser, NATIONAL STARCH AND CHEMICAL COMPANY, 10 Finderne Avenue, Bridgewater, NJ, 08807-0500, US

(21) Appl. No.: 10/298,813

(22) Filed: Nov. 18, 2002

Related U.S. Application Data
(60) Provisional application No. 60/380,508, filed on May 14, 2002.

(51) Int. Cl. A61K 31/716; A61K 9/48; A61K 9/20; D04H 1/00; B27N 3/00

(52) U.S. Cl. 424/465; 514/60; 264/109

(57) ABSTRACT

This patent pertains to a tablet comprising as a binder a low amylose starch, which has been fully debranched using isoamylase and the method of making such tablet. Such binders are useful in any tabletting method, including direct compression, and can be used as a replacement for microcrystalline cellulose.

### BACKGROUND OF THE INVENTION

The present invention relates to the use of completely linear short chain alpha-glucans as a pharmaceutical excipient, particularly as a directly compressible filler and binder, with good binding and disintegration properties and solid dosage forms containing such starch.

Solid dosage forms such as tablets and capsules usually consist of several inert materials, referred to as excipients, in addition to the active ingredient, which is present in amounts sufficient to accomplish the desired pharmaceutical effect. These excipients are generally classified according to their functions, such as fillers (also called bulking agents and diluents), binders which hold the ingredients together, binder-fillers which perform both functions and disintegrants which help the tablet to break apart and release the active ingredient when placed in a fluid environment.

Manufacture of solid dosage forms, such as tabletting and capsule-filling operations, are commonly based on the ability of certain powders to bind under compression. Typical direct compression binders include microcrystalline cellulose, compressible sugars, specific calcium salts, lactose, and dextrose. Of these, microcrystalline cellulose is the preferred binder and displays good disintegration properties. However, tablets made with this binder tend to have significant weight variations due to poor flow and low bulk density. Also microcrystalline cellulose is very expensive. Other preferred binders include the directly compressible calcium phosphates (di- or tribasic), compressible sugars, and directly compressible lactose (anhydrous and monohydrate), but each has its disadvantage. Namely, the calcium salts do not allow one to prepare tablets with a high level of active ingredient, tend to have uneven surfaces, require higher compression force to achieve target hardness, have high levels of chemical incompatibility with various drugs and generally require the use of disintegrants in high concentrations. The sugars (mostly made up of sucrose) present a darkening problem, tend to increase in hardness with age, and may react with drugs. Lactose exhibits a browning reaction with various amino drugs and also when exposed to heat and moisture; it also requires the use of a disintegrant. Mannitol and sorbitol have certain taste advantages, but either lack binding properties, require a disintegrant or are too hygroscopic; and the presence of reducing sugars often causes drug instability and are expensive.

Starch excipients are known in the art. U.S. Pat. No. 6,010,717 discloses a tabletting excipient based on disintegrated starch granules characterized by at least 10% long-chain amylose, a cold-water solubility of at most 25% and a specific area of at least 1 m²/g. U.S. Pat. Nos. 5,585,114 and 5,629,018 disclose delayed release dosage forms containing a polysaccharide matrix which consists of crystalline straight chained glucans (amylose). These patents make use of amylose containing starches.

Other starch excipients are known in the art which may use a low amylose containing starch as a base. For example, WO 97/31627 discloses microcrystalline starch as a tabletting excipient, wherein the microcrystalline starch is produced using a “starch-splitting” enzyme, that is an endo-enzyme. This patent does not use starch-debranching enzymes.

Further starch excipients are known in the art which use starch debranching enzymes. U.S. Pat. No. 5,468,286 discloses a process for preparing a tablet excipient by enzymatically treating a starch containing greater than 90% amylopectin with an alpha,-1,6-D-glucanohydrolase to partially debranch the starch and yield a mixture comprising amylopectin, partially debranched amylopectin and combinations thereof.

None of these starches display all of the desirable binder properties of microcrystalline cellulose in direct compression tabletting. Due to the high cost of microcrystalline cellulose, there is a need for compressible starches which are suitable for use as binders in any tabletting method, particularly direct compression.

Surprisingly, it has now been discovered that completely linear, short chain alpha-1,4-glucans which are highly crystallized provide an excellent replacement for microcrystalline cellulose as a binder-filler in solid dosage forms, including in direct compression tablets, and have superior disintegration properties.

### SUMMARY OF THE INVENTION

This patent pertains to a solid dosage form comprising as a binder-filler a low amylose containing starch which has been fully debranched using isoamylase and the method of making such dosage form. Such excipients are useful in any tabletting method, including direct compression, providing excellent binding, flow and filling properties. The starch excipients can be used as a total or partial replacement for microcrystalline cellulose in a tablet dosage form or can be used in combination with other non-microcrystalline cellulose directly compressible excipients and have excellent dissolution properties

As used herein, the term dosage form is intended in its broadest sense to mean not only pharmaceutical dosage forms which employ excipients to deliver active agent(s) and includes tablets (such as immediate release, controlled release, modified release, and effervescent), capsules, pellets, and granules, but also non-pharmaceutical forms of these products.

Excipient, as used herein includes binders, fillers, and all other ingredients which are pharmacologically inert.

As used herein, the term short chain amylose refers to linear polymers containing from about 5 to 65 anhydroglucose units linked by alpha-1,4-D-glucoside bonds.

Fully or completely debranched starch, as used herein, is intended to mean that which theoretically comprises 100%, by weight, of short chain amylose and, in practice, that which is so highly debranched that further enzyme activity produces no measurable change in the percentage of short chain amylose.

### BRIEF DESCRIPTION OF THE FIGURES

FIG. 1 depicts Heckel plots for microcrystalline cellulose (Avicel® PH 102) and debranched starch binders.

FIG. 2 depicts dissolution profiles for Amitriptyline tablets.

FIG. 3 depicts dissolution profiles for Caffeine tablets.

### DETAILED DESCRIPTION OF THE INVENTION

This patent pertains to a solid dosage form comprising as a binder-filler a low amylose containing starch which has been fully debranched using isoamylase and the method of making such dosage form. Such binder-fillers are useful in any tabletting method, including direct compression, providing excellent binding and filling properties as well as excellent dissolution properties.

Starch, as used herein, is intended to include all starches derived from any native source, any of which may be suitable for use herein. A native starch as used herein, is one as it is found in nature. Also suitable are starches derived from a plant obtained by standard breeding techniques including crossbreeding, translocation, inversion, transformation or any other method of gene or chromosome engineering to include variations thereof. In addition, starch derived from a plant grown from artificial mutations and variations of the above generic composition, which may be produced by known standard methods of mutation breeding, are also suitable herein.

Typical sources for the starches are cereals, tubers, roots, legumes and fruits. The native source can be waxy varieties of corn (maize), pea, potato, sweet potato, banana, barley, wheat, rice, oat, sago, amaranth, tapioca (cassava), arrowroot, canna, and sorghum, particularly maize, potato, cassava, and rice. As used herein, the term “waxy” or “low amylose” starch is intended to include a starch containing no more than about 10% by weight amylose. Particularly suitable in the invention are those starches which contain no more than about 5% amylose by weight.

The starch is completely hydrolyzed by isoamylase or another debranching enzyme capable of achieving complete hydrolysis. The enzymatic hydrolysis of the starch base is carried out using techniques known in the art. The amount of enzyme used is dependent upon the enzyme source and activity and base material used. Typically, the enzyme is used in an amount of from about 0.05 to about 2%, particularly from about 0.1 to about 0.4%, by weight of the starch.

The optimum parameters for enzyme activity will vary depending upon the enzyme used. The rate of enzyme degradation depends upon factors known in the art, including the enzyme concentration, substrate concentration, pH, temperature, the presence or absence of inhibitors, and the degree and type of modification if any. These parameters may be adjusted to optimize the digestion rate of the starch base.

The starch is gelatinized using techniques known in the art before isoamylase hydrolysis. Techniques known in the art include those disclosed for example in U.S. Pat. Nos. 4,465,702, 5,037,929, 5,131,953, and 5,149,799. Also see, Chapter XXII—“Production and Use of Pregelatinized Starch”, Starch: Chemistry and Technology, Vol. III—Industrial Aspects, R. L. Whistler and E. F. Paschall, Editors, Academic Press, New York 1967. The gelatinization process unfolds the starch molecules from the granular structure, thereby permitting the enzyme to more easily and uniformly degrade the starch molecules.

The starches may also be converted and include without limitation fluidity or thin-boiling starches prepared by oxidation, acid hydrolysis, enzyme hydrolysis, heat and or acid dextrinization. These processes are well known in the art.

Generally the enzyme treatment is carried out in an aqueous or buffered slurry at a starch solids level of about 10 to about 40%, depending upon the base starch being treated. A solids level of from about 15 to 35% is particularly useful, from about 18 to 30% more particularly useful, in the instant invention. In the alternative, the process may utilize an enzyme immobilized on a solid support.

Typically, enzyme digestion is carried out at the highest solids content feasible without reducing reaction rates in order to facilitate any desired subsequent drying of the starch composition. Reaction rates may be reduced by high solids content as agitation becomes difficult or ineffective and the starch dispersion becomes more difficult to handle.

The pH and temperature of the slurry should be adjusted to provide effective enzyme hydrolysis. These parameters are dependent upon the enzyme to be used and are known in the art. In general, a temperature of about 25 to about 70° C. is used, particularly from about 50 to about 60° C. In general, the pH is adjusted to about 3.0 to about 6.0, particularly from about 3.5 to about 4.5, using techniques known in the art.

The enzyme reaction is continued until the starch is completely debranched. In general, the enzyme reaction will take from about 1 to about 24 hours, particularly about 4 to about 12 hours. The time of the reaction is dependent upon the type of starch used, the amount of enzyme used, and the reaction parameters of solids percent, pH, and temperature.

The amount of hydrolysis may be monitored and defined by measuring the concentration of reducing groups which are freed by alpha-1,6-D-glucanohydrolase activity by methods well known in the art. Other techniques such as monitoring the change in viscosity, iodine reaction, or the change in molecular weight may be used to define the reaction end point. When the starch is completely debranched, the monitored measurement will no longer change. Typically, the starch will be completely debranched when it has been at least about 95%, more particularly at least about 98%, most particularly at least about 99% debranched by weight. The debranched starch will typically have an average chain length of 14-25 glucose units and less than about 0.2%, particularly less than about 0.1% alpha-1,6-D-glucosidic bonds (linkages).

Optionally, the enzyme may be deactivated (denatured) by any technique known in the art such as heat, acid or base deactivation. For example, acid deactivation may be accomplished by adjusting the pH to lower than 3.0 for at least 30 minutes or heat deactivation may be accomplished by raising the temperature to from about 80 to about 90° C. and maintaining it at that temperature for at least about 20 minutes to fully deactivate the enzyme.

The starch may also be further modified, either before or after the enzymatic hydrolysis. Such modification may be physical, enzyme, or chemical modification. Physical modification includes by shearing or thermally inhibiting, for example by the process described in U.S. Pat. No. 5,725,676.

Chemical modification includes without limitation, crosslinking, acetylation and organic esterification, hydroxyalkylation, phosphorylation and inorganic esterification, cationic, anionic, nonionic, and zwitterionic modifications, and succination. Such modifications are known in the art, for example in Modified Starches: Properties and Uses, Ed. Wurzburg, CRC Press, Inc., Florida (1986).

Any starch base having suitable properties for use herein may be purified by any method known in the art to remove starch off flavors and colors that are native to the polysaccharide or created during processing. Suitable purification processes for treating starches are disclosed in the family of patents represented by EP 554 818 (Kasica, et al.). Alkali washing techniques are also useful and described in the family of patents represented by U.S. Pat. Nos. 4,477,480 (Seidel) and 5,187,272 (Bertalan et al.). Such purification methods are also useful on the debranched starch.

The resultant solution is typically adjusted to the desired pH according to its intended end use. In general, the pH is adjusted to from about 5.0 to about 7.5, particularly from about 6.0 to about 7.0, using techniques known in the art. Further, any short chain amylose which precipitated out of the starch dispersion may be redispersed.

If purification of the debranched starch composition is desired, reaction impurities and by-products may be removed by dialysis, filtration, centrifugation or any other method known in the art for isolating and concentrating starch compositions. For example, the degraded starch may be washed using techniques known in the art to remove soluble low molecular weight fractions, such as oligosaccharides, resulting in more highly crystalline starch.

The debranched starch is allowed to crystallize by methods known in the art, for example by allowing the starch to stand and retrograde. The starch is then recovered using methods known in the art, particularly by filtration, centrifugation, or drying, including spray drying, freeze drying, flash drying or air drying, more particularly by filtration or flash drying. The particle size of the dried powder may be adjusted using methods known in the art including, without limitation, by agglomeration. The particle size of the dried powder is controlled during manufacture by methods known in the art to obtain an average (mean) particle size of at least about 25 microns, particularly at least about 30 microns, more particularly at least about 40 microns, and no more than about 90 microns.

Optionally, the moisture content may be adjusted to allow for improved flow and compaction. It is important to control the crystallization, typically by controlling retrogradation and drying, in order to obtain the high degree of crystallinity essential to the present invention. It is further important that the method of drying and other post-crystallization processes do not substantially destroy the crystals.

A particularly useful embodiment is one in which the starch is debranched at a low solids level, particularly at a solids level of about 5 to about 25%, more particularly about 10 to about 20%, by weight. Such low solids levels allows a larger mean crystal particle size, particularly at least about 10 microns, more particularly at least about 25 microns, most particularly at least about 40 microns, and no more than about 80 microns, as measured by the Horiba process described, infra. Crystal particle size is intended to mean the particle size in aqueous solution.

The resulting starch is in the form of highly crystalline short chain amylose from the debranched starch and is uniquely functional as a pharmaceutical excipient. The starch is characterized by a peak melting temperature, Tp, as measured by DSC using the procedure described infra, of at least about 90° C., more particularly at least about 100° C., most particularly at least about 110° C. The starch is also characterized by an enthalpy, ΔH, as measured by DSC using the procedure described infra, of at least about 25 J/g, more particularly at least about 30. Such DSC values are indicative of the highly crystalline nature of the product.

The debranched starch is typically characterized by a dextrose equivalent (DE) of at least about 5.0, more particularly of at least 6.0, most particularly at least about 7.0. However, a lower dextrose equivalent (e.g. a DE of at least about 4.0) may be achieved by altering the processing conditions, particularly by removing the low molecular weight hydrolysis products.

Dextrose equivalent, as used herein, is intended to mean the reducing power of the hydrolysate. Each starch molecule has one reducing end; therefore DE is inversely related to molecular weight. The DE of anhydrous D-glucose is defined as 100 and the DE of unhydrolyzed starch is virtually zero.

The starch is even further characterized by a bulk density of at least about 0.3 g/ml, more particularly at least about 0.4 g/ml and no more than about 0.7 g/ml.

The starch is uniquely functional as a pharmaceutical excipient in that it not only acts as a binder-filler, but also results in a solid dosage form which has excellent dissolution properties. Such starch allows for good compressibility and hardness of a tablet, which may be prepared by direct compression. In general the hardness of a compact tablet made with 100 percent of the debranched starch at 20001 lbs (8896.4 N) is at least about 20, more particularly at least about 30, most particularly at least about 38 kilopascals (kP), as measured by the methodology described in Example 3, infra.

The resultant binder-filler also provides excellent flow for direct compression which is important to obtain the desired weight of the tablet, for obtaining content uniformity of the active agent, to prevent segregation and for manufacturing efficiency. Using the binder-filler of the present invention, an angle of repose of less than about 25 degrees, particularly less than about 30 degrees, may be achieved.

The starch is used in dosage forms at a level typical in the art, particularly from about 1 to about 95%, more particularly from about 1 to about 60%, most particularly from about 10 to about 50%, by weight of the tablet. The amount of binder-filler will depend on the dilution potential of the DC filler binder, the physico-chemical nature of the active agent(s), desired potency, compatibility of the components, manufacturing methods used, the dosing method used, and on the desired hardness, friability, disintegration, dissolution, and/or stability of the final tablet.

The starch may be incorporated using any of the known methods in the art for preparing such dosage forms, including direct compression.

A variety of starch compatible active agents may be employed in the tablets of this invention. The particular nature of the active ingredient is not critical, and pharmaceutical and non-pharmaceutical active ingredients, such as nutritional supplements, detergents, dyes, pesticides, agricultural chemicals, enzymes, and foods may also be employed. Typical products include without limitation capsules and tablets not only for pharmaceutical uses, but also for detergents, fertilizers, pesticides, animal feed pellets, charcoal briquettes, bouillon cubes and other food and non-food tablets.

The binder-filler of the invention is particularly useful in a compressed tablet. The compressed tablet may be made using any method known in the art, particularly by direct compression of the tablet components. In the alternative, the tablet may be prepared by dry blending the starch product with the other components of the formulation, granulating the mixture such as by fluid bed technology, roller compactor, extrusion, or high shear granulator, and dry compacting to a tablet.

Pharmaceutical excipients known in the art may be added to the pharmaceutical dosage form to impart satisfactory processing, compression, and disintegration characteristics to the formulation. Such excipients include, but are not limited to, flow enhancers, lubricants and glidants, disintegrants, colors, flavors and sweetening agents. These excipients are well known in the art and are limited only by compatibility and characteristics desired.

Lubricants and glidants include talc, magnesium stearate, calcium stearate, stearic acid, glyceryl behenate, mineral oil, polyethylene glycol, sodium stearyl fumarate, stearic acid, vegetable oil, zinc stearate, and silicon dioxide.

Disintegrants suitable for the present invention include starches, algins, gums, croscarmelose, crospovidone, sodium starch glycolate, sodium laurel sulfate, microcrystalline cellulose, polacrilin potassium, and methylcellulose.

If the final desired product is other than a pharmaceutical dosage form, alternative additives known to those arts may be present. For example, flavors and fragrances in a bath oil tablet or surfactants in a detergent tablet.

### EXAMPLES

The following examples are presented to further illustrate and explain the present invention and should not be taken as limiting in any regard. All percents used are on a weight/weight basis.

The following test procedures are used throughout the examples:

Differential scanning calorimetry—Differential scanning calorimetry measurements were performed in a Perkin-Elmer DSC-7 (Norwalk, Conn., USA). The instrument was calibrated with indium. Samples of approximately 10 mg starch at a starch:water ratio of 1:3 are prepared and heated at 10° C./min from 5° C. to 160° C. An empty stainless steal pan is used as a reference.

Degree of Polymerization (DP)/Dextrose Equivalent (DE)—The DP or DE of the final product was determined using the Nelson/Somogyi Reducing Sugar method. See Nelson, “A photometric adaptation of the Somogyi method for the determination of glucose,” J.Biol. Chem. 153: 375-381 (1944) and Somogyi, “Notes on sugar determination,” J.Biol. Chem. 195: 19-23 (1952)

The following solutions were prepared before the tests:

Solution A: Dissolve 25 g of anhydrous sodium carbonate, 25 g of sodium potassium tartrate, and 200 g of sodium sulfate in 800 ml of deionized (D.I.) water. Dilute to 1 L, and filter if turbid.

Solution B: Dissolve 30 g of copper sulfate pentahydrate in 200 ml of D.I. water containing four drops of concentrated sulfuric acid.

Solution C: Dissolve 50 g of ammonium molybdate in 900 ml of D.I. water, and add 42 ml of concentrated sulfuric acid. Dissolve 6 g of sodium arsenate heptahydrate separately in 50 ml of D.I. water, and add this to the ammonium molybdate solution. Dilute the whole to 1 L. Warm to 55° C. to get complete dissolution if necessary.

Solution D: Add 1 ml of solution B to 25 ml of solution A.

Solution E: Dilute solution C fivefold (50 ml to 250 ml) with D.I. water.

Method:

1. Standard sugar solutions were prepared using glucose or maltose, 0.05 mg/ml, 0.1 mg/ml 0.2 mg/ml and 0.4-mg/ml. Water was used as a blank.

2. The sample solution was prepared by dissolving 0.2 g of sample to 100 ml D.I. water and boiling in a closed jar.

3. 0.5 ml of standard or sample solution was added to 0.5 ml of solution D in a microcentrifuge tube. The mixture was cooked in a boiling water bath for 20 minutes, then cooled to room temperature within 5 minutes. These solutions were vortexed for 10 seconds.

4. The mixture was transferred to 3 ml of solution E with vigorous stirring (vortex). The solutions were allowed to stand for 10 minutes and then mixed again.

5. The absorbance at 520 nm was measured.

The product DP was obtained using the standard curve plotted by the standard sugar absorbance against the concentration.

Particle Size—Median particle size of the sample was obtained with the Horiba Laser Scattering Particle Size Distribution Analyzer LA-900 (Horiba Instruments, Inc., Irvine, Calif., USA). The crystallized sample in water was added dropwise to the sample vessel of the Horiba under constant agitation until transmittance was lowered to approximately 85%. Particle size was then measured and median particle size was recorded. For the powdered final product, a sample was dilated to achieve transmittance at around 85%. The particle size was measured and median particle size was recorded.

Chain Length and Linearity—The debranched starch samples were analyzed using NMR to determine the average chain length and alpha-1,4 to alpha-1,6 linkage ratios. The NMR samples were prepared by suspending 5-6 mg of the starch in 2.5 mL of D₂O/TSP (sodium trimethyl silyl propionate) and pressure cooking the suspensions for approximately 1 hour. The resulting clear solutions were transferred to 5 mm NMR tubes and kept hot on a steam bath until the NMR spectra were acquired. This procedure for the handling of the samples insured that the crystalline starch material remained in solution. The proton NMR spectra were acquired at 90° C. on a Bruker DPX-400 spectrometer at 400 MHz.

The chemical shift assignments (relative to TSP at 90° C.) for the resonance of interest were as follows. The alpha-1,4 mid-chain linkages had a chemical shift of 5.38 ppm, the alpha-1,6 mid-chain (branch points) at 4.96 ppm, the alpha-form of the reducing end groups at 5.23 ppm, and the beta-form of the reducing end groups at 4.65 ppm.

The average chain length for the starch samples was calculated from the ratio of the reducing end groups to the mid-chain resonance. The percentage of alpha-1,6 linkages (branch points) were calculated from the amount of alpha-1,6 linkages versus alpha-1,4 linkages.

Dextrose Equivalent (DE)—For in-process DE measurement, the Fehling Volumetric Titration Method was used. Fehling solutions were prepared as follows:

A. 34.64 g of copper sulfate pertahydrate were dissolved in distilled water and diluted to 500 ml volume.

B. 173 g of potassium sodium tartrate tetrahydrate and 50 g of sodium hydroxide were dissolved in distilled water and diluted to 500 ml volume.

A 500 ml Erlenmeyer flask was rinsed with D.I. water. 50 ml of D.I. water was then added. The addition of 5 ml each of Fehling Solutions A and B, and 2 drops of methylene blue with two boiling chips followed. After determination of the reaction solids using a Refractometer, a starch solution containing 2-4 percent starch solids was prepared using D.I. water by diluting the reaction solution in a beaker. Before proceeding to the next step, the solids were checked by Refractometer to make sure the solution was prepared correctly. The beaker with starch solution was weighed and the weight recorded. 15 grams of the starch solution was added into the Erlenmeyer flask with prepared Fehlings solution. After they were boiled under agitation for 2 minutes on a hot plate, a bluish tint normally appeared. Starch solution from the beaker was added using a pipette gradually until the bluish tint disappeared and a distinctive reddish cuprous oxide formed. The starch solution was continuously stirred with plastic pipette to keep the solution uniform. When the reddish endpoint was reached, the beaker containing starch solution was weighed again to determine the weight of starch consumed. The calculation of D.E. can be seen from following equation:

$$D . E . = \frac{[ Fehling factor \times 100 ]}{[ ( grams required from starch solution ) \times ( conc . of starch solution ) ] }$$

Crushing strength—Crushing strengths were determined as an average of six (6) readings using a Pharmatron (Model 6D tablet tester, DR. Schleuniger Co., NH)

Molecular weight—Molecular weight was determined using gel permeation chromatography (GPC). A Waters 150C Gel Permeation Chromatograph configured with Viscotek, triple detection Software and hardware was used. Columns from Polymer Laboratories were used (PL gel column guard, 10⁵, 10³ and 10² angstrom columns made of highly crosslinked spherical polystyrene/divinylbenzene, 300 mm length/7.5 mm internal diameter (I.D.), 10 um packing). A mobile phase of DMSO containing 5 mM of sodium nitrate was used. Testing was completed at a flow rate of 0.7 ml/min and a temperature of 80 C.″

### Example 1

Preparation of the Crystalline Products Using Isoamylase Debranched Waxy Maize Starch

A. Two kilograms of waxy maize starch was slurried in 5.4 liters of water. The pH of the slurry was adjusted to 4.0 by adding 3:1 water:hydrochloric acid (HCI). The slurry was jet-cooked with full steam at 310-315° F. (154.4-157.2° C.) and 80 psi (5.52×10⁵ Pa) backpressure. The cooked starch solution was put into a reaction container in a 55° C. water bath. 0.2% (wt/wt) isoamylase (commercially available from Hayashibara Inc. Japan) based on starch was added to start the debranching reaction. Reaction conditions were maintained at 55° C. and pH 4.0 during the entire reaction.

After the reaction proceeded for 5 hours, the pH was adjusted to 5.5 using a 3% solution of sodium hydroxide. The isoamylase enzyme was then denatured by heating the sample to 85-90° C. in a boiling water bath for 20 minutes. The sample was cooled to room temperature and agitated at room temperature (25° C.) overnight (16 hours). The product was filtered to produce a starch cake and air-dried. The product had a degree of polymerization (DP) of 15 using Nelson/Somogyi reducing sugar test and gave a type-B x-ray diffraction pattern.

B. The method of Example 1A was repeated with the exception that the sample was cooled to 40° C. and held at 40° C. overnight for the crystallization instead of at room temperature. The product gave a type-A x-ray diffraction pattern.

C. The method of Example 1A was repeated with the exception that the sample was crystallized at 4° C.

D. The method of Example 1A was repeated with the exception that the reaction time was allowed to proceed for 24 hours instead of 5 hours. The product had a D.P. of 14 and gave a type-A x-ray diffraction pattern.

A GPC study demonstrated that all four samples were more than 95% debranched.

The DSC results for samples 1A, 1B, and 1D are shown in Table 1.

TABLE 1

| | Onset | Peak | | |
|---|---|---|---|---|
| Sample | (° C.) | (° C.) | End (° C.) | ΔH (J/g) |
| 1A | 62.1 | 89.4 | 103.7 | 26.3 |
| 1B | 66.6 | 95.8 | 119.8 | 40.0 |
| 1D | 92.7 | 115.7 | 133.4 | 28.7 |

### Example 2

Preparation of the Crystalline Starch Product Using Low Solid Reaction

1.8 kg of waxy maize starch was slurried in 5.4 liter of water. The sample was jet-cooked with full steam at 310-315° F. (154.4-157.2° C.) and 80 psi (5.52×10⁵ Pa) backpressure. The cooked starch solution was diluted to 10% solid and put into a reaction container at 55° C. The sample pH was adjusted to 4.0 by adding 3:1 water:HCI. The sample temperature was maintained at 55° C. and 0.2% isoamylase was added to start the debranching reaction. After sample DE reached 7.5 (about 8 hours), the pH was decreased to 2.0 for 30 minutes to denature the enzyme, and then increased to 6.0 using 3% sodium hydroxide. The sample was cooled to room temperature and allowed to crystallized overnight (16 hours). A sample cake was obtained by filtration and the sample was air-dried.

Tablet hardness of the sample was studied using the following test. The sample was coarsely ground and screened using US #40 mesh (opening of 0.420 mm). 600 mg of the pass-through material was weighed and compressed on the single punch tablet press. 100% binder at 2000 lbs (8896.4 N) compression force and 2-3 second compaction time resulted in a tablet crushing strength of 37 kP. This demonstrates that the resultant crystallized material has good tablet hardness.

### Example 3

Tabletting and Tablet Characterization Studies

A. Binder Compressibility Analysis Study

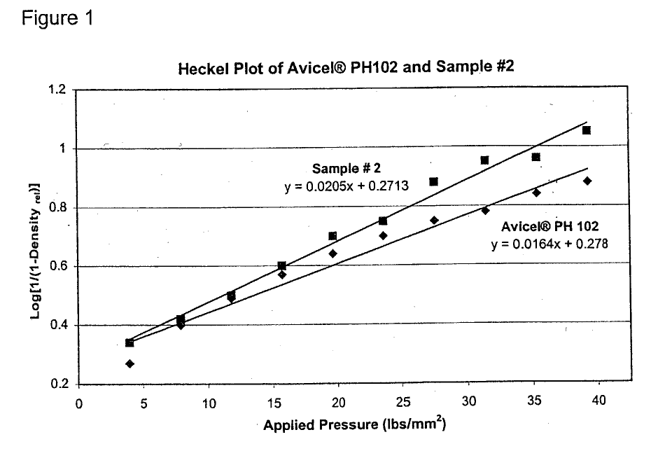

A Heckel plot allows interpretation of the binder bonding mechanism. Thus a Heckel plot was obtained for each binder according to the following methodology. A single punch tablet press machine and a ½″ flat faced punch and corresponding die were used for this study. Approximately 600 mg binder was fed into the die cavity and compressed at 500, 1000, 1500, 2000, 2500, 3000, 3500, 4000, 4500 and 5000 lbs compression force (2224.1, 4448.2, 6672.3, 8896.4, 11120.6, 13344.7, 15568.8, 17792.9, 20017.0, 22241.1 Newtons, respectively). FIG. 1 shows the Heckel plot for binders, using:

$$log \frac{1}{E} = kP + A$$

where, P=Applied pressure, E=Porosity at applied pressure P, and k=Constant related to the yield value of the powder.

The slope of Sample 2 is larger than that of Avicel® PH 102, which clearly shows that the compressibility of Sample 2 as a binder is better than Avicel® PH 102.

B. Pure Binder Compression Study

Each 100% binder was compressed by a single punch tablet press machine. Approximately 600 mg binder was fed into a ½″ die cavity and compressed at 2000 lbs compression force (8896.4 N). Crushing strength was determined using a Pharmatron (Model 6D tablet tester, DR. Schleuniger Co., NH).

| | | Standard Deviation |
|---|---|---|
| Sample | Average (kP) | (kP) |
| Avicel ® PH 102 | 38.40 | ±1.90 |
| Sample 1D | 34.40 | ±2.70 |

C. Dilution Potential Studies:

Binder dilution with non-compressible excipient (dicalcium phosphate) was performed using the four samples prepared above. The binders were blended with dicalcium phosphate to yield 3:1 excipient:binder powder blend. The powder blends were compressed by single punch tablet press at a compression force of 2000 lbs. (8896.4 N) and at the 600-mg tablet weight. Table 2 summarizes the tablet hardness for the binder samples prepared above and the industrial standard microcrystalline cellulose (MCC, Avicel PH102, FMC Corporation, Lot No. 2813).

TABLE 2

| Sample | % Moisture | Tablet Hardness (kP) |
|---|---|---|
| MCC (Avicel PH102) | — | 7.5 ± 1.0 |
| 1A | 12 | 12.0 ± 1.0 |
| 1B | 7.1 | 8.5 ± 1.0 |
| 1C | 8.7 | 11.0 ± 1.0 |
| 1D | 10.4 | 9.5 ± 1.0 |

As can be seen from Table 2, all four samples performed better as a binder than microcrystalline cellulose, resulting in harder tablets.

### Example 4

Preparation of the Crystalline Product from Acid Converted Waxy Maize Starch

A. 500 lbs. (227 kg) of an acid converted waxy maize starch was slurried in 1500 lbs. (681 kg) of water. The pH was adjusted to 4.0 using 3:1 water:hydrochloric acid. The starch was steam-batch-cooked. 0.2% isoamylase enzyme was added under constant agitation after the cooked starch temperature was cooled to 55° C. After the reaction proceeded for 8 hours, sample D.E leveled off at 6.5. At this point, the isoamylase enzyme was denatured by lowering pH to 2.0 at 55° C. for 30 minutes. The starch solution was then cooled to room temperature after pH was re-adjusted to 6.0, and allowed to crystallize at room temperature until the filtrate soluble leveled off (12 hours). The crystallized product was de-watered by centrifugation and flash-dried.

B. The method of Example 4A was repeated with the exception that the starch was an acid converted waxy maize starch with a water fluidity of 80. The final D.E. leveled off at 7.0 and after the crystallization, the product was de-watered and flash-dried.

GPC study indicated that molecular weights of these two samples were very similar although different base materials were used. The crushing strength of 100% binder at 2000 lbs. (8896.4 N) for tablets made from samples 4A and 4B were determined. Results are shown in Table 3 together with DSC data.

TABLE 3

| | Tablet | DSC | | | |
|---|---|---|---|---|---|
| Sample | hardness (kp) | To(° C.) | Tp(° C.) | Tc(° C.) | ΔH(J/g) |
| 4A | 21.9 @ 7.2% M | 47.2 | 96.2 | 127.7 | 32.0 |
| 4B | 37.4 @ 8.2% M | 84.0 | 109.0 | 122.8 | 33.2 |
| | 23.9 @ 6.1% M | | | | |

### Example 5

Preparation of the Crystalline Product Using Isoamylase Debranched Waxy Potato Starch

1 kg of waxy potato starch was slurried in 4 liters of water. The sample was jet-cooked with full steam at 310-315° F. (154.4-157.2° C.) and 80 psi (5.52×10⁵ Pa) backpressure. The cooked starch solution was put into a reaction container in a 55° C. water bath. pH was adjusted to 4.0 by adding 3:1 water:HCI. After the temperature of the sample was cooled to 55° C., 0.2% isoamylase was added. The reaction was allowed to proceed overnight (16 hours), then pH was increased to 5.5 using 3% sodium hydroxide. The sample was next heated to 85-90° C. in a boiling water bath for 30 minutes to denature the enzyme. After the enzyme was denatured, the sample was crystallized overnight at room temperature, filtered, and air-dried. The sample was then screened to get 75 to 250 μm particle sizes. The DSC results for the sample are shown in Table 4.

TABLE 4

| | Onset | Peak | | |
|---|---|---|---|---|
| Sample | (° C.) | (° C.) | End (° C.) | ΔH (J/g) |
| sample 5 | 62.2 | 96.6 | 115.3 | 36.2 |

A tablet hardness study for the sample demonstrated that, at 100% binder, compression force 2000 lbs. (8896.4 N) and the 600 mg tablet weight, the tablet hardness of the debranched and crystallized waxy potato sample was equal to the industrial standard Avicel PH102 (MCC from FMC Corporation). At 25% binder and 75% non-compressible excipient (dicalcium phosphate), the tablet hardness of the debranched and crystallized waxy potato sample was slightly better than the Avicel PH102. Results are summarized in Table 5.

TABLE 5

| | Tablet Hardness (kP) at | Tablet Hardness (kP) at |
|---|---|---|
| Sample | 100% binder | 25% binder |
| Avicel PH102 | 38.5+/−1.0 | 7.5+/−0.5 |
| Sample 5 | 38.0+/−0.8 | 8.4+/−0.3 |

### Example 6

Comparison of Isoamylase and Pullulanase Debranched Crystalline Starches

A. Two kilograms of waxy maize starch was slurried in 5.4 liters of water. The pH of the slurry was adjusted to 5.5 by adding 3:1 water:hydrochloric acid (HCI). The slurry was jet-cooked with full steam at 310-315° F. (154.4-157.2° C.) and 80 psi (5.52×10⁵ Pa) backpressure. The cooked starch solution was put into a reaction container in a 55° C. water bath. 9.6% (wt/wt) pullulanase (commercially available from Novo Nordisk) based on starch was added to start the debranching reaction. Reaction conditions were maintained during the entire reaction.

After reaction proceeded for 48 hours, the funnel viscosity was 7.5 seconds. 80 parts of magnesium sulfate (MgSO4.7H₂0) was added to help precipitate out the starch. The sample was cooled to room temperature and agitated at room temperature (25° C.) overnight (16 hours). The product was filtered to produce a starch cake and dried.

B. The binders of Example 1A, 1B, 1D, and 6A were compressed at a compression force of 2000 lbs. (8896.4 N) after blending with dicalcium phosphate in a ratio of 3:1. The tablets were compressed using a rotary Piccola press and a 3/16 inch (4.8 mm) tooling at a target weight of 300 mg.

TABLE 6
Hardness Test Data (300 mg 3/16″ (4.8 mm) flat-faced tablets,
compression force 2000 lbs (8896.4 N))

| | | Average | |
|---|---|---|---|
| | Sample | (kP) | Standard Deviation (kP) |
| 1 | Avicel ® PH 102 | 7.75 | ±0.80 |
| 2 | Sample 1A | 18.32 | ±1.02 |
| 3 | Sample 1B | 14.98 | ±0.56 |
| 4 | Sample 1D | 17.80 | ±0.65 |
| 5 | Sample 6A | 7.78 | ±1.01.70 |

As can be seen from Table 6, the samples prepared using isoamylase debranched starch (Samples 1A, 1B, and 1C) are significantly harder than those using microcrystalline cellulose or pullulanase debranched starch (Sample 6A).

### Example 7

Tablet Formulation Compositions

A. Caffeine Tablets

Caffeine, direct compression (DC) binder sample 1D, lactose anhydrous, compressible sugar, Ac-Di-Sol (Crosscarmelose sodium, Manufactured by FMC corporation), and Cab-O-Sil (Fumed silicon dioxide, Cabot Corporation) were blended in the Turbula mixer for 5 minutes, then sieved through a US#40 mesh (sieve opening of 0.420 mm) and blended for another 10 minutes. Stearic acid was sieved through a US#40 mesh, added to the mixture, and blended for 3 more minutes. Then magnesium stearate was sieved through a US#40 mesh, added to the mixture and blended for additional 3 minutes.

A Piccola 10-station press with ⅜ flat beveled edge punch and corresponding die was used to compress the tablets. The results are shown in Table 7, below.

Formulation Composition of Caffeine Tablets

Weight %

| Ingredient | Function | 6A1 | 6A2 |
|---|---|---|---|
| Caffeine USP, Spectrum | Model Drug | 46.39 | 46.39 |
| (Chemical Mfg. Corp) | | | |
| Avicel PH 102 | DC Binder | 18.00 | |
| Sample 1D | DC Binder | | 18.00 |
| Lactose Anhydrous, DC | Filler/Diluent | 17.08 | 17.08 |
| Grade (Quest International) | | | |
| Compressible Sugar | Filler/Diluent | 16.53 | 16.53 |
| (Domino Sugar Corp) | | | |
| Cab-O-Sil (Cabot | Glidant | 0.50 | 0.50 |
| Corporation) | | | |
| Ac-Di-Sol (FMC) | Disintegrant | 1.00 | 1.00 |
| Stearic Acid (Spectrum | Lubricant | 0.25 | 0.25 |
| Chemical Mfg. Corp) | | | |
| Magnesium Stearate | Lubricant | 0.25 | 0.25 |
| (Witco Corp.) | | | |

TABLE 7
Crushing Strength for Caffeine Tablets Containing Avicel PH 102 and
Sample 1D

| Tablet | Avicel ® PH 102 | Sample 1D |
|---|---|---|
| Avg. Hardness | 14.68 kP | 15.99 kP |
| Std. Dev. | 0.7696 | 0.7447 |

B. Amitriptyline Tables

Amitriptyline hydrochloride, Sample 1D as a DC binder, lactose anhydrous, Ac-Di-Sol, Cab-O-Sil were weighed and mixed in the Turbula mixer for 5 minutes. After the mixture was screened through a #40 mesh, mixed for additional 10 minutes. Then stearic acid was screened through a #40 mesh and added, the whole batch was mixed in the Turbula mixer for another 2 minutes.

After formulation, the tablets were compressed by a Piccola 10-station press with ¼ standard concave punch and corresponding die. The results are shown in Table 8, below.

Formulation Composition of Amitriptyline Hydrochloride Tablets

Weight %

| Ingredient | Function | 6B1 | 6B2 |
|---|---|---|---|
| Amitriptyline Hydrochloride | Model Drug | 9.09 | 9.09 |
| USP, (Spectrum Chemical | | | |
| Mfg. Corp) | | | |
| Avicel PH 102 | DC Binder | 15.00 | |
| Sample 1D | DC Binder | | 15.00 |
| Lactose Anhydrous DC | Filler/Diluent | 73.16 | 73.16 |
| grade (Quest International) | | | |
| Ac-Di-Sol (FMC corp.) | Disintegrant | 2.00 | 2.00 |
| Cab-O-Sil (Cabot Corp.) | Glidant | 0.25 | 0.25 |
| Magnesium Stearate | Lubricant | 0.50 | 0.50 |
| (Witco Corp.) | | | |

TABLE 8
Crushing Strength of Amitriptyline Hydrochloride Tablets
Containing Avicel PH 102 and Sample 1D

| Tablet | Avicel ® PH 102 | Sample 1D |
|---|---|---|
| Avg. Hardness | 6.205 kP | 7.685 kP |
| Std. Dev. | 0.332 | 0.534 |

Hardness data from two examples of formulation containing active drugs show that the debranched starch excipient has superior or similar binding properties to microcrystalline cellulose and can produce hard tablets.

### Example 8

Dissolution of Tablets

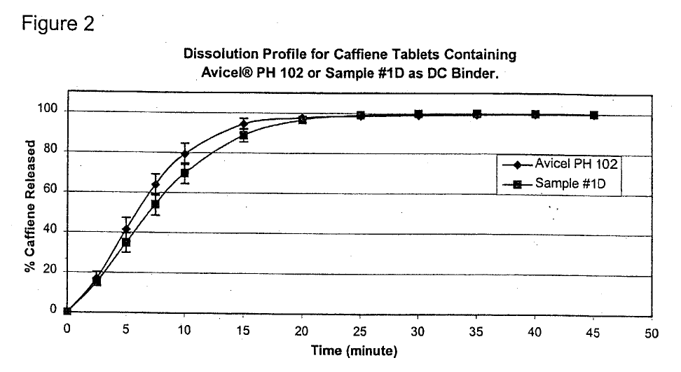

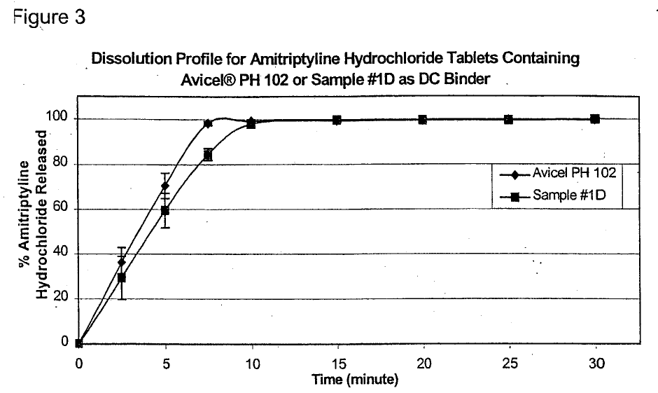

The dissolution test followed USP 24 guidelines. An USP Type 2 dissolution apparatus (Model Premiere 5100, Distek, NJ) was used in the test. This equipment was connected to a UV/Vis spectrophotometer (Model HP 8453, Hewlett Packard, Germany) equipped with eight 0.1 cm flow cells, via a 8-channel peristaltic pump (Model HP 89092A, Hewlett Packard, Germany). The percentage of drug released at predetermined time intervals was calculated and plotted against the sampling time to obtain the release profile. The dissolution profiles for the tablets of Example 7 are shown in FIGS. 2 and 3.

In this study, the functionality of debranched starch as a direct compression binder was evaluated by comparing its performance with commercially available microcrystalline cellulose (MCC). To obtain similar tablet weight and hardness, the required tabletting compression forces for debranched starch and MCC are similar. In some cases, debranched starch containing tablets showed even better tabletting performance. In general, weight variation is very small for all tablets. In the case of the Amitriptyline tablets, debranched starch containing tablets showed lower weight variation than the MCC containing tablets.

Generally, hardness variation is low for all tablets. Most debranched starch containing tablets exhibited higher hardness strength and variation than MCC containing tablets.

All tablets could release almost 100% drug within 45 minutes.

We claim:

1. A solid dosage form comprising a binder-filler and at least one active agent, said binder-filler consisting essentially of a starch composition comprising highly crystalline, fully debranched linear α-glucans, wherein the starch composition is characterized by
a) a dextrose equivalent greater than about 4.0;
b) a peak melting temperature, Tₚ as measured by DSC, of at least about 90;
c) an enthalpy, ΔH as measured by DSC of at least about 25;
d) an average particle size of at least about 25 microns and no more than about 90 microns.

2. The dosage form of claim 1, wherein the starch composition is prepared by the method comprising:
a) fully debranching a low amylose starch using isoamylase;
b) allowing the debranched starch to crystallize into crystals; and
c) drying the highly crystalline debranched starch to obtain a starch composition with an average particle size of at least about 25 microns and no more than about 90 microns.

3. The dosage form of claim 2, wherein the mean particle size of the crystals is at least about 30 microns.

4. The dosage form of claim 2, wherein the mean particle size of the crystalline starch is at least about 40 microns.

5. The dosage form of claim 2, wherein the low amylose starch comprises at least 95% amylopectin by weight.

6. The dosage form of claim 1, wherein the dextrose equivalent of the starch composition is greater than about 5.0.

7. The dosage for of claim 1, wherein the dextrose equivalent of the starch composition is greater than about 6.0.

8. The dosage form of claim 1, wherein the bulk density of the starch composition is at least about 0.3 g/ml.

9. The dosage form of claim 8, wherein the bulk density of the starch composition is at least about 0.4 g/ml and no more than about 0.7 g/ml.

10. The dosage form of claim 1, wherein the peak melting temperature of the starch composition is at least about 100° C.

11. The dosage form of claim 1, wherein the peak melting temperature of the starch composition is at least about 110° C.

12. The dosage form of claim 1, wherein the enthalpy of the starch composition is at least about 30 J/g.

13. The dosage form of claim 1, wherein the dosage form is a tablet.

14. The dosage form of claim 13, wherein the tablet is a pharmaceutical tablet.

15. The dosage form of claim 13, wherein the tablet is prepared by direct compression.

16. The dosage form of claim 15, wherein the tablet has a hardness of at least about 20 kP.

17. The dosage form of claim 15, wherein the tablet has a hardness of at least about 30 kP.

18. The dosage form of claim 15, wherein the tablet has a hardness of at least about 38 kP.

19. The dosage form of claim 1, wherein the starch composition has been chemically modified.

20. The dosage form of claim 1, further characterized by a mean crystal particle size of at least about 10 microns and no more than about 80 microns.

21. A method of making the dosage form of claim 1 comprising
a) gelatinizing a low amylose starch;
b) completely debranching the starch using isoamylase;
c) crystallizing the debranched starch into crystals;
d) drying the debranched starch to obtain a starch composition with a mean particle size of at least about 25 microns and no more than about 90 microns
e) adding an active agent to the starch composition to form a tablet mixture; and
f) forming the mixture into a tablet.

22. The method of claim 21, further comprising removing at least some of the low molecular weight components prior to crystallizing the debranched starch.

23. The method of claim 21, wherein the mixture is formed into a tablet using direct compression:

24. The method of claim 21, wherein the low amylose starch is selected from the group consisting of low amylose corn starch, low amylose tapioca starch, low amylose potato starch, and low amylose rice starch.

24. A method of making the dosage form of claim 20 comprising
a) slurrying a low amylose starch in an aqueous solution at a solids level of from about 5 to about 25% by weight;
b) gelatinizing the starch;
c) completely debranching the starch using isoamylase;
d) crystallizing the debranched starch into crystals;
e) drying the debranched starch to obtain a starch composition with a mean particle size of at least about 25 microns and no more than about 90 microns and a mean crystal particle size of at least about 10 microns and no more than about 80 microns;
f) adding an active agent to the starch composition to form a tablet mixture; and
g) forming the mixture into a tablet.
